(19) United States
(12) Patent Application Publication
(10) Pub. No.: US 2023/0142080 A1
(43) Pub. Date: May 11, 2023

(54) Glove for detecting multiple physiological parameters and system for detecting risk of hypertension disease

(71) Applicant: Second medical center of Chinese PLA General Hospital, Beijing (CN)

(72) Inventors: Feng CAO, Beijing (CN); Huiquan WANG, Beijing (CN)

(21) Appl. No.: 17/754,348

(22) PCT Filed: Aug. 18, 2021
(86) PCT No.: PCT/CN2021/113223

§ 371 Date: Mar. 30, 2022

(51) Int. Cl. A61B 5/00; A61B 5/1455; A61B 5/0205

(52) U.S. Cl. CPC A61B 5/6806; A61B 5/14552; A61B 5/0006; A61B 5/02055; A61B 5/7435

(57) ABSTRACT

The present disclosure provides a glove for detecting multiple physiological parameters and a system for detecting a risk of a hypertension disease. The glove includes: a main control module, an electrocardiogram (ECG) signal acquisition component and a comprehensive signal acquisition component. The ECG signal acquisition component includes multiple electrodes and an ECG acquisition module, and is configured to acquire an ECG signal. The comprehensive signal acquisition component is arranged at a position of any fingertip on an inner surface of the glove body, and is configured to obtain a pulse wave signal, and obtain blood oxygen saturation. The main control module is configured to send the obtained ECG signal, blood oxygen saturation and pulse wave signal to external equipment to determine a probability of a risk of the hypertension of the user.

### CROSS REFERENCE TO RELATED APPLICATION

This patent application is a national stage application of international Patent Application No. PCT/CN2021/113223 filed on Aug. 18, 2021, the disclosure of which is incorporated by reference herein in its entirety as part of the present application.

### TECHNICAL FIELD

The present disclosure relates to fields of a physiological signal detection technology and a hypertension risk prediction technology, and in particular to a glove for detecting multiple physiological parameters and a system for detecting a risk of a hypertension disease.

### BACKGROUND ART

Cardiovascular diseases are of sudden onset and high risk, and the hypertension is an important risk factor for the cardiovascular diseases. Hypertension refers to a comprehensive clinical feature characterized by an increase of systemic circulation arterial blood pressure (systolic and/or diastolic blood pressure) (systolic blood pressure≥140 mmHg. and diastolic blood pressure≥90 mmHg), which may be accompanied by functional or organic damage of heart, brain, and kidney. Hypertension is the most common chronic disease and the most important risk factor for cardiovascular and cerebrovascular diseases.

In order to improve the awareness rate, treatment rate and control rate of hypertension, a method provided by the conventional art is to investigate the blood pressure of the community population through questionnaire surveys, conduct primary prevention sampling and screening on survey data, and then analyze the screened data to obtain a probability of hypertension in the community population, so as to obtain the awareness rate, treatment rate and control rate of hypertension.

However, obtaining the blood pressure of the community population by using the questionnaire surveys has disadvantages such as long time consumption, high labor cost, non-objective data acquisition, and inability to continuously monitor the risk of the hypertension in the community population.

At present, a patient with hypertension needs to go to the hospital or clinic for testing to know details of his hypertension, which is not convenient for the patient to take corresponding treatment measures in time according to his hypertension level. In addition, the patient go to the hospital or clinic for testing many times within a certain period of time, which increases the cost of treatment for patient and wastes a lot of time for the patient and medical resources. Therefore, how to design a hypertension risk monitoring device needs to be solved urgently at present.

### SUMMARY

The present disclosure intends to provide a glove for detecting multiple physiological parameters and a system for detecting a risk of a hypertension disease, so as to monitor the multiple physiological parameters of a human body in real time, thereby predicting the risk of the hypertension.

To achieve the above effect, the present disclosure provides the following solutions.

A glove for detecting multiple physiological parameters includes: a main control module, a glove body, and an electrocardiogram (ECG) signal acquisition component and a comprehensive signal acquisition component arranged on the glove body.

The ECG signal acquisition component includes multiple electrodes arranged on a palm side of an outer surface of the glove body and an ECG acquisition module connected to each of the electrodes, and is configured to acquire an ECG signal of a user.

The comprehensive signal acquisition component is arranged at a position of any fingertip on an inner surface of the glove body, and is configured to:

obtain a pulse wave signal of the user by a transmission-type blood oxygen acquisition method; and

obtain blood oxygen saturation of the user by spectrophotometry and the transmission-type blood oxygen acquisition method.

The main control module is configured to obtain the ECG signal, blood oxygen saturation and pulse wave signal of the user, and send the obtained ECG signal, blood oxygen saturation and pulse wave signal of the user to external equipment to determine a probability of a risk of hypertension of the user.

A system for detecting a risk of a hypertension disease includes external equipment and the glove for detecting multiple physiological parameters.

The external equipment includes a mobile terminal and a cloud server. The mobile terminal is respectively connected to the glove for detecting the multiple physiological parameters and the cloud server through wireless communication.

The mobile terminal is configured to:

receive physiological parameter data of the user sent by the glove for detecting the multiple physiological parameters, where the physiological parameter data include the ECG signal, the blood oxygen saturation and the pulse wave signal;

obtain basic information of the user, where the basic information includes at least age, height, and gender; and

send the physiological parameter data and basic information of the user to the cloud server.

The cloud server is configured to:

determine a pulse wave transit distance based on the basic information of the user:

calculate a pulse wave velocity (PWV) based on the pulse wave signal and the pulse wave transit distance; and

predict a risk index of the hypertension disease of the user based on the PWV, the ECG signal and the blood oxygen saturation, and send the risk index of the hypertension disease to the mobile terminal.

Based on specific embodiments provided by the present disclosure, the present disclosure provides the following technical effects:

The present disclosure realizes real-time detections of the ECG information, blood oxygen saturation information and pulse wave information of the user by using a device for detecting the multiple physiological parameters in a form of the glove, so that the risk of the hypertension can be predicted at any time according to the real-time detected information, which can facilitate to guide the user in the early prevention and treatment of cardiovascular diseases.

### BRIEF DESCRIPTION OF THE DRAWINGS

To describe the technical solutions in the embodiments of the present disclosure or in the conventional art more clearly, accompanying drawings used in the embodiments will now be briefly described below. Apparently, the accompanying drawings described below are merely some embodiments of the present disclosure, and those of ordinary skill in the art may still obtain other accompanying drawings from these accompanying drawings without creative efforts.

FIG. 1 is a structural block diagram of a glove for detecting multiple physiological parameters according to the present disclosure;

FIG. 2 is a palm view of the glove for detecting the multiple physiological parameters according to the present disclosure;

FIG. 3 is a hand back view of the glove for detecting the multiple physiological parameters according to the present disclosure;

FIG. 4 is a side view of the glove for detecting the multiple physiological parameters according to the present disclosure;

FIG. 5 is a schematic structural diagram of a comprehensive signal acquisition component according to the present disclosure;

FIG. 6 is a layout diagram of various devices on the glove for detecting the multiple physiological parameters according to the present disclosure;

FIG. 7 is a structural block diagram of a system for detecting a risk of a hypertension disease according to the present disclosure; and

FIG. 8 is a diagram of an implementation process of a supervised algorithm based on a back propagation (BP) neural network according to the present disclosure.

### DETAILED DESCRIPTION OF THE EMBODIMENTS

The technical solutions of the embodiments of the present disclosure will be clearly and completely described below with reference to the accompanying drawings in the embodiments of the present disclosure. Obviously, the described embodiments are merely a part rather than all of the embodiments of the present disclosure. All other embodiments obtained by those of ordinary skill in the art on the basis of the embodiments of the present disclosure without creative effort shall fall within the scope of the present disclosure.

A main mechanism of damage to target organs such as a heart, brain, and kidney caused by the hypertension is arterial stiffness, atherosclerosis, stenosis and occlusion of blood vessels. PWV is an important index to evaluate the early arterial stiffness, and is also one of detection parameters for subclinical target organ damage.

Method for predicting a risk of hypertension according to PWV or a pulse wave transit time (PTT) include a method for calculating the PWV by using ECG signal beacons and a single-channel pulse wave to obtain the blood pressure, and a method for calculating the PWV and the PIT according to twin-channel pulse waves to obtain the blood pressure.

In view of this, the present disclosure evaluates a degree of arteriosclerosis of a user and determines a probability of a risk of a hypertension disease by the user through an artificial intelligence algorithm based on ECG information, blood oxygen saturation information and pulse wave information acquired from the user, thereby facilitating to guide the user in the early prevention and treatment of cardiovascular diseases.

To make the above intentions, features, and advantages of the present disclosure clearer and more comprehensible, the present disclosure will be further described in detail below with reference to the accompanying drawings and the specific implementations.

### Embodiment I

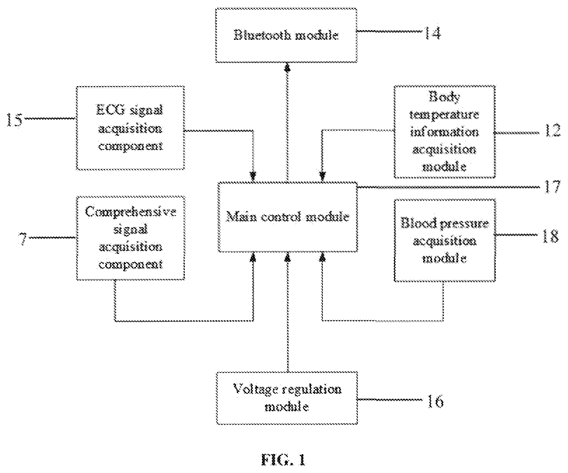

A glove for detecting multiple physiological parameters is provided in this embodiment. As shown in FIG. 1, the glove includes a main control module 17, a glove body, and an ECG signal acquisition component 15 and a comprehensive signal acquisition component both arranged on the glove body.

The ECG signal acquisition component 15 includes multiple electrodes arranged on a palm side of an outer surface of the glove body and an ECG acquisition module connected to each of the electrodes, and is configured to acquire an ECG signal of a user.

The comprehensive signal acquisition component is arranged at a position of any fingertip on an inner surface of the glove body, and is configured to:

obtain a pulse wave signal of the user by a transmission-type blood oxygen acquisition method; and

obtain blood oxygen saturation of the user by spectrophotometry and the transmission-type blood oxygen acquisition method.

The main control module 17 is configured to obtain the ECG signal, blood oxygen saturation and pulse wave signal of the user, and send the obtained ECG signal, blood oxygen saturation and pulse wave signal of the user to external equipment to determine a probability of a risk of the hypertension of the user.

Since an adult's hand is similar in size to his own heart when he clenches his fist, it is feasible to measure peripheral signals of his own heart with his own palm. The electrodes on the glove body are arranged according to standard lead distribution positions during clinical ECG monitoring. When in use, the palm wearing the detection glove is pressed on the left chest where the heart is located, and the ECG signal of the user can be acquired.

Capillaries at the fingertips are densely distributed and have a relatively obvious effect on the absorption of light in a specific wavelength range, that is, it is feasible to arrange the comprehensive signal acquisition component at a position of any fingertip of the glove body. The dark environment in the glove creates a good environment for light detection, and the human blood oxygen saturation information is acquired at the fingertip position, which can effectively avoid interference front other light.

As an optional specific implementation, the glove for detecting multiple physiological parameters provided by this embodiment further includes a body temperature information acquisition module 12. The body temperature information acquisition module 12 is arranged in a middle area of a palm of the inner surface of the glove body, and is configured to acquire a body temperature signal of the user and send the body temperature signal to the main control module 17. In use, a temperature of the palm is acquired by contact with the palm of the user to obtain accurate body temperature data.

Further, the glove for detecting multiple physiological parameters provided by this embodiment further includes a Bluetooth module 14, a voltage regulation module 16, a blood pressure acquisition module 18, and an arc wristband.

The main control module 17 is arranged on a hand back side of the inner surface of the glove body or embedded in the wristband. The Bluetooth module 14, the voltage regulation module 16, and the blood pressure acquisition module 18 are all embedded in the wristband, and move with the wristband. The main control module 17 communicates with the external equipment through the Bluetooth module 14. The blood pressure acquisition module 18 is configured to acquire a blood pressure signal of the user and send the blood pressure signal to the main control module 17. The voltage regulation module 16 includes a battery and a step-down chip connected to the battery. The step-down chip is configured to reduce a voltage output by the battery to meet power supply standards of various devices in the glove for detecting the multiple physiological parameters. The ECG signal, blood oxygen saturation, pulse wave signal, blood pressure signal and body temperature signal can be acquired simultaneously.

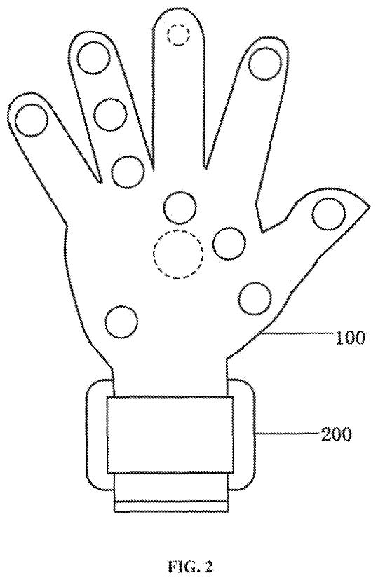

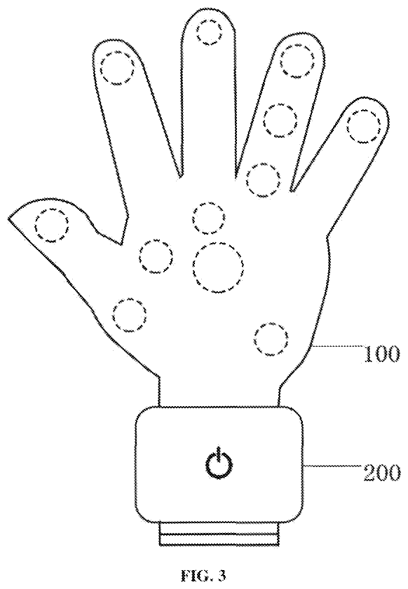

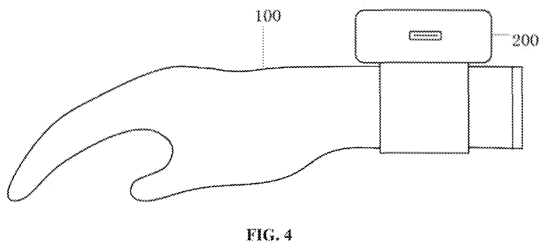

Taking wearing on a right hand as an example, the shape and structure of the glove for detecting multiple physiological parameters provided by this embodiment are shown in FIG. 2 to FIG. 4, and the glove for detecting the multiple physiological parameters includes a glove body 100 and a wristband 200. The main control module 17 is arranged on the glove body 100 or the wristband 200.

The combination of the glove body 100 and the wristband 200 is detachable, and the glove body 100 and the wristband 200 are connected through a miniHDMI plug for data interaction. A switch is arranged on the wristband 2W. The glove body 100 and the wristband 200 are made of a flexible printed circuit (FPC) to ensure the wearing comfort of the patient. The wristband is of a shape of a major arc, and can be retracted to varying degrees, so as to meet the needs of users of different ages.

Specifically, the ECG signal acquisition component 15 includes at least 10 circular electrodes, and the 10 circular electrodes are arranged on the palm side of the outer surface of the glove body according to standard lead distribution positions during clinical ECG monitoring. The 10 circular electrodes are all made of hardened silica gel.

The ECG acquisition module acquires the signal in an all-lead ECG acquisition mode. A digital analog physiological signal processing chip ADS1298 produced by T1 company based on a high-precision signal sampling method is used as the ECG acquisition module. The digital analog physiological signal processing chip ADS1298 uses a 3.3 V unipolar power supply, highly integrates 8 high-speed data conversion channels, each composed of an electro magnetic interference (EMI) filter, a programmable gain amplifier (PGA), and a 24-bit analog-to-digital converter (ADC), and also integrates commonly used functional circuits for 12-lead ECG detection such as a right leg driven circuit (RLD), a Wilson center detection circuit (WCT), and a lead-off detection circuit. Through combination with typical peripheral circuits provided in a manual, the ECG signal can be acquired more concisely.

Specifically, the blood oxygen saturation is acquired by spectrophotometry, which adopts red light with a wavelength of 660 nm and infrared light with a wavelength of 940 nm. Oxygenated hemoglobin absorbs less red light with a wavelength of 660 nm, and more infrared light with a wavelength of 940 nm, while hemoglobin does the opposite. Therefore, by determining a ratio of infrared light absorption to red light absorption by the spectrophotometry, the degree of oxygenation of hemoglobin can be determined.

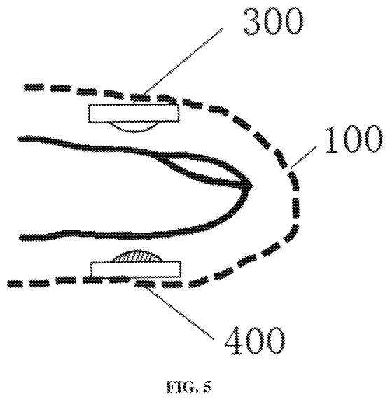

The comprehensive signal acquisition component 7 provided by this embodiment is arranged at a position of the fingertip of a middle finger on the inner surface of the glove body. Referring to FIG. 5, the comprehensive signal acquisition component 7 provided by this embodiment includes: a light emitting device 300, a photoelectric detection device 400, and a calculation module.

The light emitting device 300 is arranged on a pulp side of the middle fingertip. The light emitting device 300 includes a first light emitting diode and a second light emitting diode. The first light emitting diode is configured to emit a red light signal, the second light emitting diode is configured to emit an infrared light signal, and the first light emitting diode and the second light emitting diode can work alternately. In some embodiments, the light emitting diode is an LED device.

The photoelectric detection device 400 is arranged on a back side of the middle fingertip, and is configured to receive a calibration red light signal and a calibration infrared light signal, convert the calibration red light signal into a first electrical signal, and convert the calibration infrared light signal into a second electrical signal. The calibration red light signal is the red light signal after passing through the middle fingertip, and the calibration infrared light signal is the infrared light signal after passing through the middle fingertip. In some embodiments, the photoelectric detection device 400 is a photodiode.

The calculation module is arranged on the back side of the middle fingertip, and is configured to:

determine the pulse wave signal according to the first electrical signal; and

determine the blood oxygen saturation according to the first electrical signal and the second electrical signal.

During operation, the first light emitting diode and the second light emitting diode are turned on and off alternately, so that the photoelectric detection device 400 can distinguish light with different wavelengths, and the photoelectric detection device 400 converts the detected red light and infrared light that pass through the artery vessels of the finger into electrical signals. Since skin, muscle, fat, venous blood, pigment and bones have constant absorption coefficients for the red light and the infrared light, only concentrations of oxygenated hemoglobin HbO₂ and hemoglobin Hb in the arterial blood flow change periodically with the arteries of the blood, resulting in the periodical change of a signal intensity output by the photoelectric detection device 400. By processing these periodically changing signals, the corresponding blood oxygen saturation can be measured.

During operation, the first light emitting diode is turned on and the second light emitting diode is turned off, so that the photoelectric detection device 400 detects red light. The photoelectric detection device 400 converts the detected red light that passes through the artery vessels of the finger into an electrical signal, and then the calculation module determines the pulse wave signal according to the electrical signal.

Specifically, the body temperature information acquisition module 12 is composed of a temperature sensor LMT70 (hereinafter referred to as LMT70) combined with its peripheral circuit. LMT70 is an ultra-small, high-precision and low-power complementary metal oxide semiconductor analog temperature sensor with output enable pins. It is suitable for almost all high-precision, low-power and cost-effective temperature sensing applications, for example, medical thermometers, high-precision instruments and battery-powered devices. The heat dissipation of this sensor is less than 36 μW, and this ultra-low self-heating characteristic supports its high accuracy in a wide temperature range. LMT70 has excellent temperature matching performance, and thus a temperature difference between two adjacent LMT70s taken out of the same reel, is 0.1° C. at most. LMT70 also has a linear low-impedance output, and thus supports seamless connection with an off-the-shelf microcontroller (MCU)/ADC. Therefore, this temperature sensor has a fairly good performance in fitting with the glove.

Specifically, an STM32F407 series chip with an advanced RISC machine (ARM) as a core is used as the main control module 17, and chips with moderate performance such as STM32F407ZGT6 can be selected. A smallest system composed of STM32F4 series single-chip microcomputers is used as a control circuit for the main control module 17. The STM32F407 series chip uses 90-nanometer non-volatile memory (NVM) technology and an adaptive real-time memory accelerator (ART), and integrates new digital signal processor (DSP) and floating-point unit (FPU) instructions. The high-speed performance of 168 MHz improves an execution speed of a control algorithm and code efficiency. Furthermore, it has 1 MB of FLASH, high-speed universal synchronous asynchronous receiver transmitter (USART) up to 10.5 Mbits/s, and high-speed serial peripheral interface (SPI) up to 37.5 Mbits/s, which makes STM32F407 series chips show good accuracy and rapidity in processing the multiple physiological parameters, especially the ECG signal.

Specifically. A CC2541 chip is used as the Bluetooth module 14. The CC2541 chip supports data transfer rates of 250 Kbps, 500 Kbps, 1 Mbps, and 2 Mbps. and has excellent receiving sensitivity, powerful five-channel direct memory access (DMA), accurate digital received signal strength indicator (RSSI), eight-channel 12-bit ADC, and the chip has a configurable resolution, two powerful USART interfaces, and a 3.3 V power supply, and supports for multiple serial protocols, and an 12C interface supports rapid data exchange with the main control module 68. Accordingly, by pairing the Bluetooth module 14 connected to the main control module 68 and Bluetooth on the external equipment, wireless data transmission between them is realized, to get rid of the uncomfortable connection line surrounding the user during detection.

Specifically, the blood pressure acquisition module 18 is used in conjunction with the glove body and is equipped with a pneumatic pump and a solenoid valve.

Specifically, the voltage regulation circuit includes a 3.7 V lithium battery and a TLV70033DDCR step-down chip, and the voltage regulation circuit is configured to output a 3.3 V stable voltage. The TLV70033DCKR step-down chip has an operating temperature range of −40° C. to 150° C. It has excellent line and load transient performance, low output noise, and a very high power supply rejection ratio (PSRR) and low dropout (LDO) voltage, and thus the step-down chip is very suitable for most battery-powered handheld devices. The handheld device has a thermal shutdown function and a current limit function to ensure safety. In addition, it can be adjusted to specified accuracy without output load to meet the power supply requirements of all modules. Additionally, a charging socket is arranged to charge the lithium battery, making a power supply part of the detection glove more coordinated.

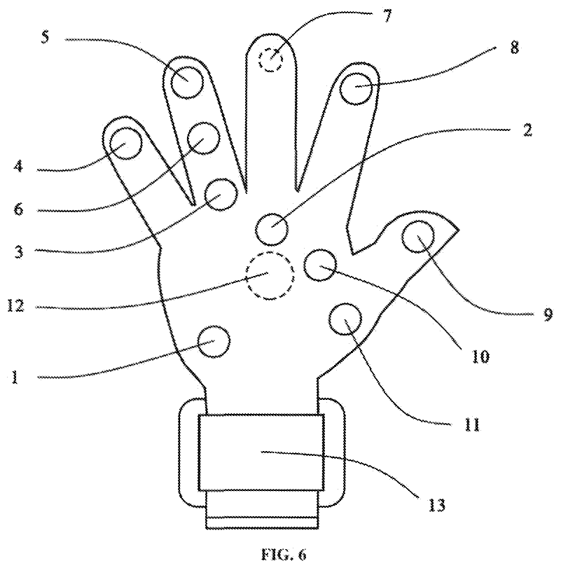

Referring to FIG. 6, the ECG signal acquisition component 71, the comprehensive signal acquisition component 7 and the body temperature information acquisition module 12 are all connected to the main control module 17 at the hand back through wires. Except for the 10 circular electrodes exposed outside, other components or modules are embedded inside the glove, which are invisible from the outside.

The 10 circular electrodes include an ECG RL lead electrode 1, an ECG V3 lead electrode 2, an ECG V4 lead electrode 3, an ECG LL lead electrode 4, an ECG V6 lead electrode 5, an ECG V5 lead electrode 6, an ECG LA lead electrode 8, an ECG RA lead electrode 9, an ECG V2 lead electrode 10, and an ECG V1 lead electrode 11. The reference numeral 7 refers to the comprehensive signal acquisition component, the reference numeral 12 refers to the body temperature information acquisition module, and the reference numeral 13 refers to a wrist blood pressure cuff.

Compared with the conventional art, the embodiments of the present disclosure has the following advantages:

First, the ECG is a means for checking the electrical activity of the heart. For example, arrhythmia, premature beats and acute myocardial infarction can all be diagnosed by the ECG. These diseases are accompanied by changes in the electrical activity of the heat. For example, for the symptom of palpitation, the ECG changes during the attack period, and the ECG can completely return to normal in the remission period. Therefore, when there is heart discomfort, such as chest tightness, palpitation, and chest pain, the first priority is not to go to a major hospital to find an expert, but to capture an ECG at the time of the onset of illness immediately, and then consult an expert after capturing the ECG at the time of the onset. ECG acquisition with ECG detection devices is the main way to check various heart diseases. The traditional ECG acquisition method uses disposable electrode pads and ECG monitors to acquire the ECG of the patient under the operation of professional medical personnel. However, this traditional ECG acquisition method needs many lead wires and is cumbersome to operate. In addition, the ECG monitor is bulky and inconvenient to move, which has a defect of delaying valuable rescue time in emergency situations. The glove for detecting the multiple physiological parameters provided by the present disclosure can solve the above-mentioned problems. In use, the ECG signal can be acquired anytime and anywhere by wearing the glove against the left chest.

Second, the blood oxygen saturation (SpO₂) is a percentage of a capacity of the oxygenated hemoglobin (HbO₂) bound by oxygen in the blood to that of all hemoglobin (Hb) that can be bound, that is, a concentration of blood oxygen in blood. The blood oxygen saturation is an important physiological parameter of a respiratory cycle, while functional oxygen saturation is a ratio of the concentration (that is, a sum of a concentration of the oxygenated hemoglobin and a concentration of the hemoglobin), which is different from the blood oxygen saturation. Therefore, monitoring arterial blood oxygen saturation can estimate the oxygen-carrying capacity of hemoglobin of the lung. The blood oxygen saturation of arterial blood in a normal human body is 98%, and the blood oxygen saturation of venous blood is 75%. A metabolic process of the human body is a biological oxidation process, and the oxygen required in the metabolic process enters the human blood through a respiratory system, combines with the hemoglobin (Hb) in the red blood cells to form oxygenated hemoglobin (HbO₂), and then is transported to various tissues and cells of the human body. Therefore, the ability to carry and transport oxygen of the blood is measured by the blood oxygen saturation. The traditional method for measuring the blood oxygen saturation is to first collect blood from the human body, then use a blood gas analyzer for electrochemical analysis to measure the partial pressure of oxygen (PO₂), and finally calculate the blood oxygen saturation based on the partial pressure of oxygen PO₂. However, this traditional method for measuring the blood oxygen saturation is cumbersome and cannot be used for continuous monitoring. A finger sleeve type photoelectric sensor is arranged in the glove for detecting multiple physiological parameters provided by the present disclosure, and during measurement, only the detection glove needs to be sleeved on the finger, the finger is then used as a transparent container for the hemoglobin, and red light with a wavelength of 660 nm and near-infrared light with a wavelength of 940 nm are used as an incident light source to measure an intensity of light transmission through a tissue bed, so as to calculate the concentration of the hemoglobin and the blood oxygen saturation. The instrument connected to the glove for detecting the multiple physiological parameters can display the human blood oxygen saturation, thereby providing a continuous and non-invasive blood oxygen measuring instrument. In addition, the finger sleeve type photoelectric sensor is embedded in the fingertip of the detection glove, and then during measurement, the dark environment in the detection glove creates a good environment for light detection, which can effectively avoid interference from other light.

Third, the body temperature refers to the temperature inside the human body. Since the temperature inside the body is not easy to measure, the temperature of the mouth, axilla or rectum is thus often used clinically to represent the body temperature. The oral temperature of a normal person is 36.7-37.7 (average 37.2), the axillary temperature is 36.0-37.4 (average 36.8), and the rectal temperature is 36.9-37.9 (average 37.5). The rectal temperature is closest to the temperature inside the human body, but it is inconvenient to measure, so the body temperature is mostly measured from the axilla and mouth. Accurate body temperature has certain reference significance for diagnosis of human diseases. At present, the most common thermometer is a glass thermometer. A mercury column in the glass thermometer can change with the body temperature, which is convenient for the user to observe at any time. Since a structure of glass is relatively dense and the performance of mercury is very stable, the glass thermometer has the advantages of accurate indication, high stability, low price, and no external power supply, and is thus deeply trusted by people, especially medical workers. However, the glass thermometer has obvious defects, for example, it is easy to break and there is the possibility of mercury pollution, and the measurement time thereof is relatively long, which is inconvenient to use for patients with acute and serious illness, the elderly, infants, etc., and the reading is cumbersome. With the development of science and technology, many new types of thermometers have emerged, such as electronic thermometers, which display the body temperature in a digital form based on a definite relationship between physical parameters of certain substances (such as resistance, voltage, and current) and the environmental temperature, thereby achieving clear readings and easy portability. The disadvantage of the electronic thermometer is that the accuracy of a display value is affected by factors such as electronic components and battery power supply status, which is not as good as the glass thermometer. The embodiment of the present disclosure can accurately, efficiently and directly acquire the human body temperature by the body temperature information acquisition module, which overcomes the above-mentioned defects.

### Embodiment II

This embodiment provides a system for detecting a risk of a hypertension disease, specifically a combination of a glove and a wristband, and the system is used in conjunction with a mobile terminal. By using the detection technology, three physiological parameters of ECG, blood oxygen saturation, and pulse wave of the user are acquired at the same time, and the acquired physiological parameters are transmitted to the mobile terminal in real time for the user to view. At the same time, the mobile terminal sends the physiological parameters and the age, height, and gender of the user to the cloud server through the Internet. As the cloud server has a built-in artificial intelligence algorithm to perform big data processing on the above data, evaluate the degree of the arteriosclerosis of the user, obtain a probability of the risk of the hypertension, and feed back to the mobile terminal for the user to view, which can facilitate to guide patients with the hypertension in the early prevention and treatment of cardiovascular diseases.

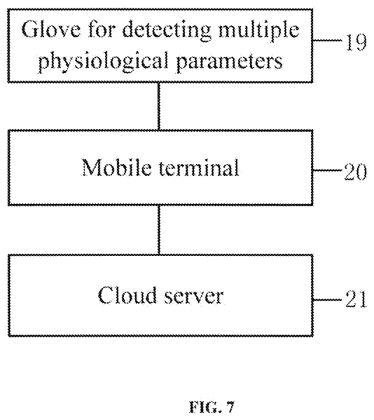

Referring to FIG. 7, a system for detecting a risk of a hypertension disease provided by this embodiment includes external equipment and the glove 19 for detecting multiple physiological parameters described in the Embodiment 1.

The external equipment includes a mobile terminal 20 and a cloud server 21. The mobile terminal 20 is respectively connected to the glove 19 for detecting the multiple physiological parameters and the cloud server 21 through wireless communication.

The mobile terminal 20 is configured to:

receive physiological parameter data of the user sent by the glove 19 for detecting the multiple physiological parameters, where the physiological parameter data include the ECG signal, the blood oxygen saturation and the pulse wave signal;

obtain basic information of the user, where the basic information includes at least age, height, and gender; and

send the physiological parameter data and basic information of the user to the cloud server 21.

The cloud server 21 is configured to:

determine a pulse wave transit distance based on the basic information of the user;

calculate a PWV based on the pulse wave signal and the pulse wave transit distance; and

predict a risk index of the hypertension disease of the user based on the PWV, the ECG signal and the blood oxygen saturation, and send the risk index of the hypertension disease to the mobile terminal 20.

The cloud server is described in more detail below.

The cloud server has a built-in neural network model for predicting the pulse wave transit distance. The neural network model for predicting the pulse wave transit distance is composed of a BP neural network, and is configured to predict a distance between the brachial artery and the heart in different groups, that is, the pulse wave transit distance. Therefore, the function of the neural network model for predicting the pulse wave transit distance is to predict the pulse wave transit distance corresponding to a current user according to the basic information input by the current user.

Therefore, during determining the pulse wave transit distance based on the basic information of the user, the cloud server is configured to determine the pulse wave transit distance of the user based on the basic information of the user and the neural network model for predicting the pulse wave transit distance. The neural network model for predicting the pulse wave transit distance is determined according to a physical feature sample and a BP neural network. The physical feature sample includes multiple sets of basic information of calibration users and tag information corresponding to each set of the basic information of the calibration users. The tag information is the pulse wave transit distance of the calibration user.

The PTT is calculated based on pulse wave information, and then the PWV is calculated according to a formula (1).

$$PWV = \frac{D}{PTT} ; \qquad ( 1 )$$

where D represents the pulse wave transit distance.

Therefore, during calculating the PWV based on the pulse wave signal and the pulse wave transit distance, the cloud server is configured to determine the PTT based on the pulse wave information, and determine a ratio of the pulse wave transit distance to the PTT as the PWV.

The cloud server has a built-in relationship curve of calibration physiological parameter data-degree of arteriosclerosis-risk of hypertension. The number of the curves is not limited to one, and the curves are not for a specific age group, height group, or gender group, but are multiple mapping relationships and related empirical formulas established for different age groups, users of different heights, and different gender groups.

Therefore, during predicting the risk index of the hypertension disease of the user based on the PWV, the ECG signal and the blood oxygen saturation, and sending the risk index of the hypertension disease to the mobile terminal, the cloud server is configured to:

calculate a percentage of a degree of arteriosclerosis and the risk index of the hypertension based on the PWV, the ECG signal, the blood oxygen saturation, and the relationship curve of calibration physiological parameter data-degree of arteriosclerosis-risk of hypertension.

Specifically, the BP neural network performs supervised learning on the learning results of sparse auto-encoding to achieve fine-tuning of network parameters. The BP neural network includes an input layer, a Mini-batch layer, a full connection layer, a hidden layer, an encoding layer, an output layer, and an iteration layer. In this embodiment, the height, age, and gender information of 8000 people and the length of the brachial artery (i.e., pulse wave transit distance) of each person are used as physical feature samples for BP neural network training and prediction respectively.

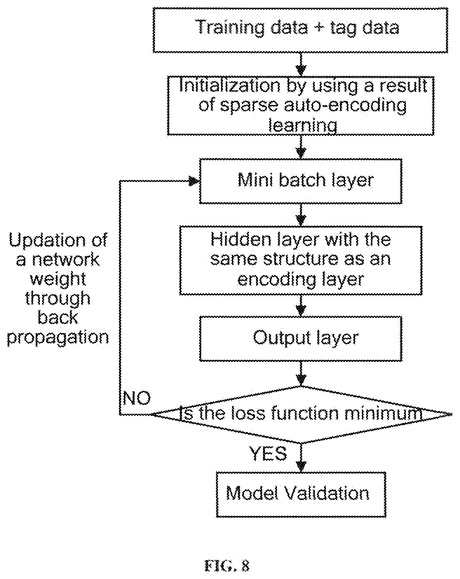

The input layer uses 80% of the physical feature samples as a training data set with a dimension of N×Len. 80% of a data set of the length of the brachial artery is used as tag data with a dimension ofN×Len. 20% of the physical feature samples is used as validation data with a dimension of 0.25N×Len. 20% of the data set of the length of the brachial artery is used as control data with a dimension of 0.25N×Len. The mini-batch layer is divided into batches according to the size of p′ one batch contains p′ groups of physical feature information, and there are 0.4N/p′ batches in total. The physical feature information in each batch enters the full connection layer one by one, that is, the dimension of entering the hidden layer is 1×Len, There are the same number of the hidden layers and the encoding layers, and each hidden layer has the same number of neurons. A weight result of the sparse auto-encoding learning is used as an initialization parameter of the BP neural network. A physical feature matrix of the output layer has a dimension of 1×Len. A physical feature matrix Ŷ with a dimension of p′×Len is output after p′ groups of physical features all pass through the full connection layer. The iteration layer is to construct a loss function, and update the weight through a BP theorem, so that the output after physical feature conversion has a higher similarity to the corresponding length of the brachial artery. After a batch of physical features is traversed, the loss function is calculated once, and the network weight is updated once. After all batches of physical feature data are traversed, one iteration is completed, and the loss function J′ is as follows:

$${J}^{′} = \frac{1}{{p}^{′}} {\sum }_{h = 1}^{{p}^{′}} {( {\hat{y}}_{h} - {y}_{h} )}^{2} ; \qquad ( 2 )$$

where ŷₕ is an output result after conversion of an h-th group of physical features, ŷₕ is control data of an h-th group of data of the length of the brachial artery, that is, the predicted length of the brachial artery, and p′ is the number of physical feature data in a batch. The flow of the algorithm in this section is shown in FIG. 8.

The mobile terminal receives data from the Bluetooth module 14, transmits the received data to the cloud server for analysis, and then displays them completely and in real time. The mobile terminal is described in more detail below.

The mobile terminal has a built-in APP software. The APP software includes an obtaining module, an input module, an interface display module, and an output module.

The obtaining module is configured to obtain the physiological parameter data of the user sent by the glove for detecting the multiple physiological parameters through wireless communication, and specifically, the APP software establishes connection with the glove for detecting the multiple physiological parameters by turning on the Bluetooth function of the mobile terminal, and receives physiological parameter data acquired by the glove for detecting the multiple physiological parameters via the Bluetooth function.

The obtaining module is further configured to obtain the percentage of the degree of the arteriosclerosis and the risk index of the hypertension sent by the cloud server through wireless communication.

The input module is configured to obtain the basic information of the user, and specifically, the APP software has a built-in algorithm program, which is configured to firstly require the user to enter his height, age, and gender information or confirm the stored height, age, and gender information every time the user opens the APP software.

The output module is configured to send the physiological parameter data and basic information of the user to the cloud server through wireless communication.

The interface display module is configured to display the twelve-lead ECG, a heart rate, a pulse wave, blood oxygen saturation, the percentage of the degree of the arteriosclerosis, and the risk index of the hypertension of the user. The twelve-lead ECG and heart rate are determined according to the ECG signal.

The above system for detecting the risk of the hypertension disease is specifically used in the following steps.

(1) The mobile terminal needs to be connected to the Internet. The Bluetooth is turned on. The APP software is opened to wait for pairing with the Bluetooth of the glove for detecting multiple physiological parameters. After successful pairing, the user is prompted to enter height, age, and gender information. (2) After the user wears the glove body and the wristband on his right hand, a switch in the middle of the hand back is pressed to wait for successful connection between the Bluetooth and the mobile terminal. (3) Five fingers of the palm are stretched and pressed on the left chest in a direct contact with the skin without clothing, and it shall be ensured that the center of the palm is located in the middle of the breastbone, and the height is flush with the heart. The thumb is stretched toward the upper right, the index finger is stretched toward the upper left, and the angle of the right hand is adjusted to ensure that the fingertip of the thumb is at the same height as the fingertip of the index finger. The middle and ring fingers are naturally stretched to ensure that a natural arc is formed from the root of the thumb to the fingertip of the ring finger, and the fingertip of the ring finger is as close to the left anterior axillary line as possible. The little finger is stretched toward the lower left to ensure that the fingertip of the little finger is on a midline connecting the fingertips of the thumb and index fingers. (4) The ECG acquisition module acquires the original ECG signals. The comprehensive signal acquisition component acquires the blood oxygen saturation and the pulse wave information. The above three original physiological parameters are amplified and filtered by the main control module and converted into data packets with communication protocols, and finally valid data is transmitted to the mobile terminal via the Bluetooth. (5) The mobile terminal uploads the data to the cloud server in real time, and the cloud server processes the data and then feeds it back to the mobile terminal for display. (6) The mobile terminal displays and saves the received ECG data in real time in a form of a graphical user interface (GUI), which is not only convenient for patients to view their own conditions, but also for doctors to diagnose and analyze the real-time ECG and historical ECG of the monitored person. The mobile terminal displays feedback results from the cloud server in real time.

The present disclosure has the following beneficial effects:

Advantage 1: The acquired ECG signals are accurate, and 10 specific parts of the palm are selected as the lead acquisition positions. The signals around the heart are strong and can reflect the conditions of various chambers of the heart.

Advantage 2: The efficiency of ECG acquisition is high. Using the glove as a carrier of ECG acquisition is more convenient than clinically using large-scale equipment and the electrode pads, and the acquisition efficiency is higher.

Advantage 3: The acquired blood oxygen saturation is accurate. The dark environment in the glove creates a good environment for light detection. Acquiring the human blood oxygen information at the position of the middle finger can effectively avoid the interference from other light.

Advantage 4: Body temperature detection is more convenient and fast. Clinically, methods such as mercury thermometers, electronic thermometers, and forehead thermometers are generally used to detect the core temperature of the human body. Such methods are easily affected by the external environment, the measurement time is long and the subject needs to sit still or lie flat during temperature measurement. It is more convenient and fast to acquire the body temperature information in the form of the glove.

Advantage 5: Use and operation are simple. During operation, the user only needs to turn on the switch on the hand back and puts on the glove close to the left chest, and the user can view its own physiological parameters on the mobile terminal.

Advantage 6: The comfort of the subject is improved. Compared with the traditional method of acquiring the ECG with the electrode pads, the glove is light and thin, and thus the temperature of the glove in use can be less different from that of the chest, which makes the subject feel more comfortable than the cold electrode pads.

Advantage 7: The whole machine is small and light. In the clinical monitoring environment, the doctor generally use a multi-physiological parameter monitor provided by the hospital to obtain various physiological parameters of a postoperative patient or an emergency patient. Such a device is bulky and inconvenient to move, and must be operated by medical personnel such as doctors and nurses in the hospital as the device has a complicated circuit and cumbersome operation. The whole machine of the present disclosure is small and light, and easy to operate, and is not only suitable for emergency situations, but also suitable for monitoring the physiological parameters of the postoperative patients or home situations.

Advantage 8: The portable detection system for predicting early hypertension disease provided by the present disclosure can quickly and accurately detect twelve-lead ECG information, blood oxygen information, and body temperature data of the human body in real time and in a portable way, and a PWV value can be obtained according to the above information and the risk of the hypertension of the user can be predicted. In terms of predicting the risk of the hypertension, it not only saves the time spent on investigating the physical signs and basic information of the user, which saves labor costs, but also can predict the risk level of the hypertension before the user is out of health, which is convenient for real-time monitoring of the physical conditions of the user.

The embodiments of this specification are described in a progressive manner, each embodiment focuses on the difference from other embodiments, and the same and similar parts among the embodiments may refer to each other. Specific embodiments are used to expound the principle and implementations of the present disclosure. The description of these embodiments is merely used to assist in understanding the method of the present disclosure and its core conception. In addition, those of ordinary skill in the art can make modifications in terms of specific implementations and scope of application based on the conception of the present disclosure. In conclusion, the content of this specification shall not be construed as a limitation to the present disclosure.

What is claimed is:

1. A glove for detecting multiple physiological parameters, comprising: a main control module, a glove body, and an electrocardiogram (ECG) signal acquisition component and a comprehensive signal acquisition component both arranged on the glove body;
the ECG signal acquisition component comprises a plurality of electrodes arranged on a palm side of an outer surface of the glove body and an ECG acquisition module connected to each of the electrodes, and is configured to acquire an ECG signal of a user;
the comprehensive signal acquisition component is arranged at a position of any fingertip on an inner surface of the glove body, and is configured to:
obtain a pulse wave signal of the user by a transmission-type blood oxygen acquisition method; and
obtain blood oxygen saturation of the user by spectrophotometry and the transmission-type blood oxygen acquisition method; and
the main control module is configured to obtain the ECG signal, blood oxygen saturation and pulse wave signal of the user, and send the obtained ECG signal, blood oxygen saturation and pulse wave signal of the user to external equipment to determine a probability of a risk of hypertension of the user.

2. The glove for detecting multiple physiological parameters according to claim 1, wherein the ECG signal acquisition component comprises at least ten circular electrodes, and the ten circular electrodes are arranged on the palm side of the outer surface of the glove body according to standard lead distribution positions during clinical ECG monitoring; and
the ECG acquisition module acquires the signal in an all-lead ECG acquisition mode.

3. The glove for detecting multiple physiological parameters according to claim 1, wherein the comprehensive signal acquisition component is arranged at the position of the fingertip of a middle finger on the inner surface of the glove body;
the comprehensive signal acquisition component comprises a first light emitting diode, a second light emitting diode, a photoelectric detection device, and a calculation module;
the first light emitting diode arranged on a pulp side of the fingertip of the middle finger is configured to emit a red light signal;
the second light emitting diode arranged on the pulp side of the fingertip of the middle finger is configured to emit an infrared light signal;
the photoelectric detection device arranged on a back side of the fingertip of the middle finger is configured to receive a calibration red light signal and a calibration infrared light signal, convert the calibration red light signal into a first electrical signal, and convert the calibration infrared light signal into a second electrical signal; wherein the calibration red light signal is the red light signal after passing through the fingertip of the middle finger, and the calibration infrared light signal is the infrared light signal after passing through the fingertip of the middle finger; and
the calculation module arranged on the hack side of the fingertip of the middle finger is configured to:
determine the pulse wave signal according to the first electrical signal; and
determine the blood oxygen saturation according to the first electrical signal and the second electrical signal.

4. The glove for detecting multiple physiological parameters according to claim 1, further comprising a Bluetooth module, a voltage regulation module, a blood pressure acquisition module, and an are wristband;
the main control module is arranged on a hand back side of the inner surface of the glove body or embedded in the wristband; and the Bluetooth module, the voltage regulation module, and the blood pressure acquisition module are all embedded in the wristband;
the main control module communicates with the external equipment via the Bluetooth module;
the blood pressure acquisition module is configured to acquire a blood pressure signal of the user and send the blood pressure signal to the main control module; and
the voltage regulation module comprises a battery and a step-down chip connected to the battery; and the step-down chip is configured to reduce a voltage output by the battery to meet power supply standards of various devices in the glove for detecting the multiple physiological parameters.

5. The glove for detecting multiple physiological parameters according to claim 1, further comprising a body temperature information acquisition module; and
the body temperature information acquisition module is arranged in a middle area of a palm of the inner surface of the glove body, and is configured to acquire a body temperature signal of the user and send the body temperature signal to the main control module.

6. A system for detecting a risk of a hypertension disease, comprising external equipment and a glove for detecting multiple physiological parameters,
wherein
the glove for detecting multiple physiological parameters, comprising: a main control module, a glove body, and an electrocardiogram (ECG) signal acquisition component and a comprehensive signal acquisition component both arranged on the glove body;
the ECG signal acquisition component comprises a plurality of electrodes arranged on a palm side of an outer surface of the glove body and an ECG acquisition module connected to each of the electrodes, and is configured to acquire an ECG signal of a user;
the comprehensive signal acquisition component is arranged at a position of any fingertip on an inner surface of the glove body, and is configured to:
obtain a pulse wave signal of the user by a transmission-type blood oxygen acquisition method; and
obtain blood oxygen saturation of the user by spectrophotometry and the transmission-type blood oxygen acquisition method; and
the main control module is configured to obtain the ECG signal, blood oxygen saturation and pulse wave signal of the user, and send the obtained ECG signal, blood oxygen saturation and pulse wave signal of the user to external equipment to determine a probability of a risk of hypertension of the user;
the external equipment comprises a mobile terminal and a cloud server;
the mobile terminal is respectively connected to the glove for detecting the multiple physiological parameters and the cloud server through wireless communication;
the mobile terminal is configured to:
receive physiological parameter data of the user sent by the glove for detecting the multiple physiological parameters, wherein the physiological parameter data comprise the ECG signal, the blood oxygen saturation and the pulse wave signal;
obtain basic information of the user, wherein the basic information comprises at least age, height, and gender; and
send the physiological parameter data and basic information of the user to the cloud server; and
the cloud server is configured to:
determine a pulse wave transit distance based on the basic information of the user;
calculate a pulse wave velocity (PWV) based on the pulse wave signal and the pulse wave transit distance; and
predict a risk index of the hypertension disease of the user based on the PWV, the ECG signal and the blood oxygen saturation, and send the risk index of the hypertension disease to the mobile terminal.

7. The system for detecting the risk of the hypertension disease according to claim 6, wherein during determining the pulse wave transit distance based on the basic information of the user, the cloud server is configured to:
determine the pulse wave transit distance of the user based on the basic information of the user and a neural network model for predicting the pulse wave transit distance, wherein the neural network model for predicting the pulse wave transit distance is determined according to physical feature samples and a back propagation (BP) neural network; the physical feature samples comprise multiple sets of basic information of calibration users and tag information corresponding to each set of the basic information of the calibration users; and the tag information is the pulse wave transit distance of the calibration users.

8. The system for detecting the risk of the hypertension disease according to claim 6, wherein during calculating the PWV based on the pulse wave signal and the pulse wave transit distance, the cloud server is configured to:
determine a pulse transit time (PTT) based on pulse wave signal; and
determine a ratio of the pulse wave transit distance to the PTT as the PWV.

9. The system for detecting the risk of the hypertension disease according to claim 6, wherein during predicting the risk index of the hypertension disease of the user based on the PWV, the ECG signal and the blood oxygen saturation, and sending the risk index of the hypertension disease to the mobile terminal, the cloud server is configured to:
calculate a percentage of a degree of arteriosclerosis and the risk index of the hypertension based on the PWV, the ECG signal, the blood oxygen saturation, and a relationship curve of calibration physiological parameter data-degree of arteriosclerosis-risk of hypertension.

10. The system for detecting the risk of the hypertension disease according to claim 9, wherein APP software is provided in the mobile terminal;
the APP software comprises an obtaining module, an input module, an interface display module, and an output module; wherein
the obtaining module is configured to:
obtain the physiological parameter data of the user sent by the glove for detecting the multiple physiological parameters through wireless communication; and
obtain the percentage of the degree of the arteriosclerosis and the risk index of the hypertension sent by the cloud server through wireless communication;
the input module is configured to obtain the basic information of the user:
the output module is configured to send the physiological parameter data and the basic information of the user to the cloud server through wireless communication; and
the interface display module is configured to display a twelve-lead ECG, a heart rate, a pulse wave, blood oxygen saturation, the percentage of the degree of the arteriosclerosis, and the risk index of the hypertension of the user; and the twelve-lead ECG and the heart rate are determined according to the ECG signal.

11. The system for detecting the risk of the hypertension disease according to claim 6, wherein the ECG signal acquisition component comprises at least ten circular electrodes, and the ten circular electrodes are arranged on the palm side of the outer surface of the glove body according to standard lead distribution positions during clinical ECG monitoring; and
the ECG acquisition module acquires the signal in an all-lead ECG acquisition mode.

12. The system for detecting the risk of the hypertension disease according to claim 11, wherein during determining the pulse wave transit distance based on the basic information of the user, the cloud server is configured to:
determine the pulse wave transit distance of the user based on the basic information of the user and a neural network model for predicting the pulse wave transit distance, wherein the neural network model for predicting the pulse wave transit distance is determined according to physical feature samples and a back propagation (BP) neural network; the physical feature samples comprise multiple sets of basic information of calibration users and tag information corresponding to each set of the basic information of the calibration users; and the tag information is the pulse wave transit distance of the calibration users.

13. The system for detecting the risk of the hypertension disease according to claim 11, wherein during calculating the PWV based on the pulse wave signal and the pulse wave transit distance, the cloud server is configured to:
determine a pulse transit time (PTT) based on pulse wave signal; and
determine a ratio of the pulse wave transit distance to the PTT as the PWV.

14. The system for detecting the risk of the hypertension disease according to claim 11, wherein during predicting the risk index of the hypertension disease of the user based on the PWV, the ECG signal and the blood oxygen saturation, and sending the risk index of the hypertension disease to the mobile terminal, the cloud server is configured to:
calculate a percentage of a degree of arteriosclerosis and the risk index of the hypertension based on the PWV, the ECG signal, the blood oxygen saturation, and a relationship curve of calibration physiological parameter data-degree of arteriosclerosis-risk of hypertension.

15. The system for detecting the risk of the hypertension disease according to claim 11, wherein APP software is provided in the mobile terminal;
the APP software comprises an obtaining module, an input module, an interface display module, and an output module; wherein
the obtaining module is configured to:
obtain the physiological parameter data of the user sent by the glove for detecting the multiple physiological parameters through wireless communication; and
obtain the percentage of the degree of the arteriosclerosis and the risk index of the hypertension sent by the cloud server through wireless communication;
the input module is configured to obtain the basic information of the user;
the output module is configured to send the physiological parameter data and the basic information of the user to the cloud server through wireless communication; and
the interface display module is configured to display a twelve-lead ECG a heart rate, a pulse wave, blood oxygen saturation, the percentage of the degree of the arteriosclerosis, and the risk index of the hypertension of the user; and the twelve-lead ECG and the heart rate are determined according to the ECG signal.

16. The system for detecting the risk of the hypertension disease according to claim 6, wherein the comprehensive signal acquisition component is arranged at the position of the fingertip of a middle finger on the inner surface of the glove body;
the comprehensive signal acquisition component comprises a first light emitting diode, a second light emitting diode, a photoelectric detection device, and a calculation module;
the first light emitting diode arranged on a pulp side of the fingertip of the middle finger is configured to emit a red light signal;
the second light emitting diode arranged on the pulp side of the fingertip of the middle finger is configured to emit an infrared light signal;
the photoelectric detection device arranged on a back side of the fingertip of the middle finger is configured to receive a calibration red light signal and a calibration infrared light signal, convert the calibration red light signal into a first electrical signal, and convert the calibration infrared light signal into a second electrical signal; wherein the calibration red light signal is the red light signal after passing through the fingertip of the middle finger, and the calibration infrared light signal is the infrared light signal after passing through the fingertip of the middle finger; and
the calculation module arranged on the back side of the fingertip of the middle finger is configured to:
determine the pulse wave signal according to the first electrical signal; and
determine the blood oxygen saturation according to the first electrical signal and the second electrical signal.

17. The system for detecting the risk of the hypertension disease according to claim 16, wherein during determining the pulse wave transit distance based on the basic information of the user, the cloud server is configured to:
determine the pulse wave transit distance of the user based on the basic information of the user and a neural network model for predicting the pulse wave transit distance, wherein the neural network model for predicting the pulse wave transit distance is determined according to physical feature samples and a back propagation (BP) neural network; the physical feature samples comprise multiple sets of basic information of calibration users and tag information corresponding to each set of the basic information of the calibration users; and the tag information is the pulse wave transit distance of the calibration users.

18. The system for detecting the risk of the hypertension disease according to claim 16, wherein during calculating the PWV based on the pulse wave signal and the pulse wave transit distance, the cloud server is configured to:
determine a pulse transit time (PT) based on pulse wave signal; and
determine a ratio of the pulse wave transit distance to the PIT as the PWV.

19. The system for detecting the risk of the hypertension disease according to claim 6, wherein the glove for detecting multiple physiological parameters further comprises a Bluetooth module, a voltage regulation module, a blood pressure acquisition module, and an arc wristband;
the main control module is arranged on a hand back side of the inner surface of the glove body or embedded in the wristband; and the Bluetooth module, the voltage regulation module, and the blood pressure acquisition module are all embedded in the wristband;
the main control module communicates with the external equipment via the Bluetooth module;
the blood pressure acquisition module is configured to acquire a blood pressure signal of the user and send the blood pressure signal to the main control module; and
the voltage regulation module comprises a battery and a step-down chip connected to the battery; and the step-down chip is configured to reduce a voltage output by the battery to meet power supply standards of various devices in the glove for detecting the multiple physiological parameters.

20. The system for detecting the risk of the hypertension disease according to claim 6, wherein the glove for detecting multiple physiological parameters further comprises a body temperature acquisition module; and
the body temperature information acquisition module is arranged in a middle area of a palm of the inner surface of the glove body, and is configured to acquire a body temperature signal of the user and send the body temperature signal to the main control module.
